(19) United States
(12) Patent Application Publication
(10) Pub. No.: US 2002/0127625 A1
(43) Pub. Date: Sep. 12, 2002

(54) Methods of diagnosing immune related diseases

(75) Inventor: Vivi-Anne Oxelius, Lund (SE)

Correspondence Address: FINNEGAN, HENDERSON, FARABOW,, GARRETT and DUNNER, L.L.P, 1300 I Street, N.W., Washington, DC, 20005-3315, US

(73) Assignee: FORSKARPATENT IS SYD AB, Lund (SE)

(21) Appl. No.: 10/099,162

(22) Filed: Mar. 18, 2002

Related U.S. Application Data
(63) Continuation of application No. 09/541,050, filed on Mar. 31, 2000.

(51) Int. Cl. G01N 33/53; G01N 33/537; G01N 33/543

(52) U.S. Cl. 435/7.92

(57) ABSTRACT

A method for in vitro determination, of allelic alternative genetic markers for the IgG subclasses IgG1, IgG2 and IgG3 from chromosome 14q32 in serum of a patient, wherein the expression of the activated IgG1, IgG2 and IgG3 genes is determined, differentiating 4 genetically different B-cells, is described. There is also described a kit for use in such a method.

### FIELD OF INVENTION

This invention relates to diagnosis and research of allergic diseases, primary immunodeficiency diseases and autoimmune diseases.

### BACKGROUND OF THE INVENTION

Human IgG is the biggest immunoglobulin class in serum and most important for the immune defence. IgG consists of four isotypes, namely IgG subclasses IgG1, IgG2r IgG3 and IgG4, deviating immunochemically and functionally. The immunoglobulin molecule consists of two heavy and two light chains, both with a variable and a constant part. The deviating epitopes for IgG subclasses are located to the constant part of the heavy chain. For further details, see e.g. Oxelius V-A: Crossed immunoelectrophoresis and electroimmunoassay of human IgG subclasses. Acta Path Microbiol Scand. Sect A; 86:109-116, 1978, and Oxelius V-A: IgG subclass levels in infancy and childhood. Acta Paediatr Scand; 68:523-27, 1978.

### BRIEF SUMMARY OF THE INVENTION

The invention provides a method, such as a screening method, for determination in serum of allelic alternative Gm allotypes, genetic markers for IgG subclasses, both qualitatively and quantitatively. Different aspects of the invention comprise

1) qualitative determination of the allelic expression of the IGHG3, IGHG1 and IGHG2 genes on chromosome 14q32,

2) determination of 4 genetically different B cell variants according to 4 Gm haplotypes, and

3) the quantitative allelic expression from the IGHG3, IGHG1 and IGHG2 genes on chromosome 14q32,

The invention also provides a method for identification and quantification of genetically related subunits of IgG subclass molecules, which method can partly replace earlier known methods of IgG subclass determination.

In one aspect, the invention provides for a method for diagnosis of different forms of allergic diseases. In investigating bronchial asthma the genetic marker G2m(n) from IGHG2 has been proven to be in linkage disequilibrium with the high expressing IGHE gene, constituting the atopic form. The alternative genetic marker G2m(-n) from IGHG2 has been proven to be in linkage disequilibrium with the low expressing IGHE gene, that is the non atopic form.

In another aspect, the invention provides for a new in vitro test to be used early in the disease for discovery of the non atopic form of allergy. It is beside IgE determinaion a test for confirmation of the atopic form of allergy and in the same time revealing the genetic constitution, resulting in an outstanding possibility for classification of allergic diseases in which determination of IgE levels and specific IgE antibodies may be unstable and uncertain factors. In bronchial asthma, the alternative Gm allotypes have been proven to give rise to different pathways of immune regulation.

In still another aspect, the invention provides for an in vitro method of revealing different qualities (different Gm allotypes) of IgG molecules from the IGHG3, IGHG1 and IGHG2 genes on chromosome 14q32 in the atopic and non atopic forms of allergic diseases.

In yet another aspect, the invention provides for an in vitro method of revealing the quantitative expression from single alleles of the IGHG3, IGHG1 and IGHG2 genes on chromosome 14q32, expressed as serum Gm allotypes. The normal variation of serum Gm allotype quantities in healthy children and adults is given.

In a further aspect, the invention provides for an in vitro test for discovery of genetically different B cells based on the expression of the IGHG3, IGHG1 and IGHG2 genes of the alternative Gm allotypes constituting the Gm haplotypes.

In a further aspect, the invention provides for an in vitro test to be used for patients with allergy. For children it should be used early in the course of the disease to evaluate atopy or non atopy, but also as markers for prognosis as patients with homozygous variants of B cells have a more severe form of the disease.

In a still further aspect, the invention provides for an in vitro test to be used for further research in the field of allergy, immunodeficiencies and autoimmune diseases where IGH genes on chromosome 14q32 are involved.

The invention also provides for a kit for use in such methods.

### DETAILED DESCRIPTION OF THE INVENTION

A. Definitions

The terms “serum”, “patient serum”, “serum sample of a patient” as used herein are defined as any fraction of the patient's blood which contains IgG, IgG subclasses and Gm allotypes.

By “IgG subclasses IgG1, IgG2 IgG3 and IgG4” as used herein are meant subunits of IgG defined as any molecules with specific epitopes, respectively, reacting with specific polyclonal antisera produced in rabbits.

The term “Gm allotypes” as used herein means genetic subunits of the IgG subclasses IgG1, IgG2 and IgG3, that is from IgG1: G1m(f) and G1m(a), from IgG2: G2m(n) and G2m(-n), and from IgG3: G3m(b) and G3m(g). They are defined as any molecule reacting with respectively specific antibody, with the exception of G2m(-n).

The term “monoclonal antibodies” as used herein are defined as antibodies capable of binding G1m(f), G1m(a), G2m(n) and G3m(b), respectively.

The term “myeloma proteins” as used herein are defined as monoclonal serum proteins from patients with myelomatosis. The myeloma proteins are purified from serum of patients with myelomatosis. The purified myeloma proteins of type G1m(f), G1m(a), G2m(n) and G3m(b) are used, one for each test, as competitors with the naturally occurring G1m(f), G1m(a), G2m(n) and G3m(b) in serum of the patient tested.

The term “a normal serum pool” as used herein is defined as a mixture of sera from more than 200 blood donors.

By “conjugated rabbit anti-mouse immunoglobulins” as used herein is meant the immunoglobulins used for detecting the bound mouse monoclonal antibodies directed against Gm allotypes.

I. Detection of Gm Allotype Specific Molecules, Namely G1m(f), G1m(a), G2m(n), G2m(-n), G3m(b) and G3m(g)

In one of its aspects, the invention provides for methods for diagnosis of different types of allergic diseases. The B1 cell type or the haplotype Gm(bfn) is connected with the atopic form of bronchial asthma and B2 and 4 cell types or the Gm(bf-n) and Gm(ga-n) haplotypes with the non atopic form. In general the methods involve the detection of the G1m(f), G1m(a), G2m(n), G2m(-n), G3m(b) and G3m(g) Gm allotypes in serum of the patients with bronchial asthma,

More specifically, a G1m(f) molecule in the allergic patients's serum is detected by allowing the G1m(f) to interact with mouse monoclonal antibodies to G1m(f) in competition with purified myeloma proteins of type G1m(f). The interaction between G1m(f) and mouse monoclonal anti-G1m(f) is measured by adding peroxidase-conjugated rabbit anti-mouse immunoglobulins. The method can be used qualitatively and also quantitatively plotted against a curve of dilutions of a normal serum pool with known quantities of G1m(f).

The same procedure is repeated for determination of the G1m(a), G2m(n) and G3m(b) Gm allotypes, using the specific G1m (a), G2m(n) and G3m(b) myeloma proteins and anti-G1m(a), anti-G2m(n) and anti-G3m(b) mouse monoclonal antibodies, respectively.

In a preferred embodiment, the microtitre plates from Nunc-Immuno Plate Maxi Sorp and coated with predetermined concentrations of purified myeloma proteins in carbonate buffer 0.1 M, pH 9.6 overnight at +4° C. were used.

In a preferred embodiment the serum dilutions are adjusted to the amount in serum 1/20 or 1/200.

In a preferred embodiment for preparation of purified myeloma proteins, serum samples from patients with myelomatosis, presenting M-components by electrophoresis were screened and typed for the Gm allotypes, G1m(f), G1m(a), G2m(n) and G3m(b). The M-component was purified by preparative electrophoresis and further DRAE-Sepharose fractionation and, if necessary, other methods. The preparation was tested for purity with panels of antisera to IgG subclasses and monoclonal antibodies to Gm allotypes. The protein content was decided and the purified myeloma proteins were used in the test, and as positive and negative controls.

In a preferred embodiment the normal serum pool was chosen from consecutive 200 to 500 healthy blood donors, also tested for a representative frequency of Gm haplotypes from the Caucasian population. The normal serum pool was double diluted 7 times for quantitative studies.

II. The Method Further Encompasses the Determination of Serum Amounts of IgG Molecules From Different Alleles of the IGHG3, IGHG1 and IGHG2 Genes on Chromosome 14q32

III. The Method Further Encompasses the Determination of Different Genetic B-cells Based on the Gm haplotype Determination

IV. The Testing Systems can be Supplied in the Form of Kits.

### FURTHER DETAILS OF THE INVENTION

One of each IgG1, IgG2 and IgG3 subclass can be further characterized by 2 genetically related, alternative Gm allotypes for IgG1: G1m(f) and G1m(a), for IgG2 G2m(n) and G2m(-n) and for IgG3 G3m(b) and G3m(g) They represent further deviating epitopes of the heavy constant chain. With Gm allotype determinations we are studying the allelic expression from the IGHG1, IGHG2 and IGHG3 genes on chromosome 14q32.

The IgG subclass concentrations are the result of the gene activities on chromosome 14q32. Some Gm genotypes favour low IgG subclass expressions. Serum IgG and IgG subclass levels (M and SD) have been given for a normal population divided in 6 common Gm genotypes. The Gm(bf-n/bf-n)genotype expressed low IgG2 and significantly lower total IgG than other Gm genotypes. The Gm(ga-n/ga-n) genotype expressed low IgG3. There were high and low IgG subclass responding Gm genotypes, e.g. Gm(bf-n/bf-n) with especially low IgG2 and Gm(ga-n/ga-n) with especially low IgG3. New normal range levels for 1gG subclasses have been instituted originating from the Gm genotype. This was important for evaluating low levels of IgG2 and 9gG3. The influence of closely related Gm allotype genes and gene dosages was confirmed.

Quantification of the activity from the different IGHG gene loci was possible, when we developed a method for the first time for quantification of Gm allotypes. It is a sensitive competitive ELISA using monoclonal antisera and purified myeloma proteins. It is to be used together with IgG subclass quantification of IgG2 and IgG3. We have quantitated Gm allotypes in a normal population and have given the values of Gm allotypes in the 6 common Om genotypes.

With this method we can quantitate the direct allelic gene expression from the different IgG subclass gene loci for the constant heavy chain. The alternative Om allotypes of the IgG subclasses IgG1, IgG2 and IgG3 are de novo different effector molecules of the same IgG subclass differing in a few amino acid epitopes.

Quantitation of Gm allotypes has opened a new research field. The production of antibodies and activity from one or both alleles on the IgG subclass loci IGHG1, IGHG2 and IGHG3 on chromosome 14, could be studied,

By preparative electrophoresis, Protein A Sepharose gel filtration and DEAE chromatography, it was for the first time possible to identify deviating immunochemical properties of the Gm allotypes within the same IgG subclass, indicating different entities. From polyclonal IgG it was possible to separate G1m(f) from G1m(a), G2m(-n) from G2m(n) and G3m(g) from G3m(b). Purification of G2m(-n) molecules is of special interest as no genetic marker has been found to identify this allotype. An immunochemical relationship between G1m(f), G2m(n) and IgG4 was found in the anodal IgG. This is interesting also because of the close locations of the G2m(n), IgG4 and IgE genes on chromosome 14.

We could for the first time show different maturation rates of the alternative Gm allotypes within IgG1, IgG2 and IgG3 in a recent study of 430 children. The G2m(n) development from γ2 gene locus and Gm(bfn) was strikingly retarded compared to G2m(-n) from Gm(ga-n) during childhood. In stead, levels of G1m(f) from the γ1 gene locus and Gm(bfn) developed more rapidly than G1m(a) from Gm(ga-n). Different maturation rates of Gm allotypes within the same IgG subclass is a further explanation of the variation of antibody response during childhood.

Quantitation of different IgG effector molecules from the genetic variation according to Gm allotypes constitutes an additional basis for evaluation of IgG in different diseases in childhood and in vaccination programs.

Different Gm allotype amounts were unexpectedly found in commercially available human intravenous immunoglobulin (IVIG) preparations used for replacement therapy in primary and secondary immunodeficiencies, severe bronchial asthma and autoimmune disorders. Gm allotype quantities differed significantly in different IVIG products having half or double the amount of Om allotypes within the IgG subclass. This showed the effect of different manufacturing processes, but also indicated different physicochemical properties of Gm allotypes within the same IgG subclass. The different contents of Gm allotypes might be one reason for the variable levels of specific antibodies found in IVIG products.

Immunodeficient patients with homozygous expression of Gm allotypes from γ1, γ2 and γ3 gene locus were tested after IVIG infusion for foreign Gm allotypes. The consumption of Gm allotypes differed also within the same IgG subclass. A prolonged survival was found for the G2m allotype G2m(n) compared to Gm allotypes. The method is easy to use for determination of half lives of foreign Gm allotypes in individual patients and for further adjustment of the dose of IVIG.

Immunoglobulins and also Gm allotypes are unique products of B-cells. A paper was recently published (v.-A. Oxelius: Genetic B-Cell Variation Based on Immunoglobulin Heavy G-Chain (Gm) Genes. Scand. J. Immunol. 49, 345-346, 1999) about genetic B-cell variation based on immunoglobulin heavy G chain, Gm genes. Four different B-cells: B1, B2, B3 and B4 can be distinguished based on the effector molecules of the 4 different Gm haplotypes: Gm(bfn), Gm(bf-n), Gm(gan) and Gm(ga-n), respectively.

TABLE 1
Genetic variants of B cells according to Gm
haplotypes

| | Genetic code | |
|---|---|---|
| | Chromosome 14q32 | Gm |
| Genetic B cells | 5′ μ δ γ3 γ1 α1 γ2 γ4 ε α2 3′ | haplotypes |
| B1 | 5′ μ δ b f α1 n γ4 ε α2 3′ | Gm (bfn) |
| B2 | 5′ μ δ b f α1-n γ4 ε α2 3′ | Gm (bf-n) |
| B3 | 5′ μ δ g a α1 n γ4 ε α2 3′ | Gm (gan) |
| B4 | 5′ μ δ g a α1-n γ4 ε α2 3′ | Gm (ga-n) |

Different B cells produce deviating effector molecules with different amino acid epitopes of the heavy constant chain as shown by different immunochemical properties and half-life times of the Gm allotypes. The unique feature of the B cell depends on the Gm haplotypes for antibody production. The variation in a Caucasian population is the combination of the four different haplotypes resulting in 10 genotypes, some of which are common and others very rare.

TABLE 2
B cells in Caucasian individuals

| 10 Gm genotypes | Frequency | B cells |
|---|---|---|
| bfn/bfn | 22.3% | B1/B1 |
| bfn/bf-n | 20.4% | B1/B2 |
| bf-n/bf-n | 7% | B2/B2 |
| bfn/gan | 3.8% | B1/B3 |
| bfn/ga-n | 19.1% | B1/B4 |
| bf-n/gan | <1% | B2/B3 |
| bf-n/ga-n | 12.1% | B2/B4 |
| gan/gan | <1% | B3/B3 |
| gan/ga-n | 1.3% | B3/B4 |
| ga-n/ga-n | 14.0% | B4/B4 |

Different pathways of immune regulation were evident when investigating children with bronchial asthma: patients with homozygous Gm(bfn/bfn) called B1/B1 showing the atopic phenotype and the alternative Gm(ga-n/ga-n) called B4/B4 showing the non atopic phenotype. The particular B4/B4 cells found in non atopic bronchial asthma are also found in patients with immunodeficiencies. B2/B2 cells dominated in common variable immunodeficiency and IgG2 deficiency.

Serum concentrations of Gm allotypes have been determined in 100 children with atopic bronchial asthma and B1 cells, B1/B1, B1/B2 and B1/B4 expressing Gm(bfn) in homozygous or heterozygous state and compared to groups of healthy children with the same age and B1 cells. Unexpectedly it was found that the G1mf) levels from γ1 locus was significantly decreased to ¾ of the values found in the healthy group, both homozygous and heterozygous Gm(bfn). Instead the G2m(n) levels were double increased.

The expression from the upper part of the IGH gene containing G1m(f) was partly excluded, while the lower part containing G2m(n), γ4 and ε was activated. The imbalanced switch with suppressed levels of G1m(f) antibodies in atopic childhood asthma is probably the result of less exposure to infections and must be related to the increased prevalence of atopic childhood asthma in western countries the last decades. The genetic events on the IGH genes on chromosome 14q32 will lead to new diagnostic and therapeutic interventions in atopy.

### EXAMPLES

### Example 1

A sensitive competitive indirect ELISA for measuring serum concentrations of the Gm allotypes G1m(a), G1m(f), G2m(n) and G3m(b) was developed. The following monoclonal antibodies were used: anti-G1m(a) clone 5E7 (Janssen Biochimica, Belgium), anti-G1m(f) clone 5F10 (Janssen Biochimica), anti-G2m(n) clone SH21 (Sigma, St Louis, Mo, USA) and anti-G3m(b1/μ) clone 12D9 (Janseen Biochimica). Microtitre plate. (Nunc-Immuno Plate Maxi Sorp) wells were coated with 100 μl of a predetermined concentration of purified myeloma proteins of the following Gm allotypes' G1m(f), G1n(a), G2mn(n) and G3m(b) in carbonate buffer 0.1 M pH 9.6 overnight at +4° C. After two washes with PBS-Tween the wells were filled with PBS 1% BSA and incubated at room temperature for 1 h, then washed again with PBS-Tween. Thirthy (40) (50) μl of serum diluted 1/20 and 1/200 were added to the wells in duplicate. A normal serum pool in double dilutions 7 times was included in each plate in duplicate. Panels of purified myeloma proteins of the alternative Gm allotypes within the IgG subclass were used as positive and negative controls. The same volume of monoclonal antibody diluted in PBS 0.5% BSA from 1/3000 to 1/10,000 was added and mixed immediately. After incubation under continuous shaking at room temperature for 1 h, the plates were washed three times. Sixty (80) (100) μl of peroxidase-conjugated rabbit anti-mouse immunoglobulins (Dakopatts, Copenhagen, Denmark) diluted 1/1000 in PBS. Tween were added and incubated for 30 min at room temperature. After three washes bound conjugate was measured by adding 100 μl per well of OPD (Dakopatts) in phosphate-citrate buffer 0.05 M pH 5.0, 0.4 μl/ml 30% H₂O₂ as substrate. The enzyme reaction was stopped by adding 50 μl per well of 1 M H₂SO₄. Optical densities were read on a microplate reader at 450 nm (Multiskan Miss.).

The amounts (in g/l) of Gm allotypes can be calculated from the knowledge of the gene frequency in a large Caucasian population with given proportions of Gm allotypes within the IgG subclass of approximately the following: for IgG1, G1m(a)/G1m(f) 30/70, for IgG2 G2m(n)/G2m(-n) 45/55 1.50/1.83 g/l; and for IgG3, G3m(g)/G3m(b) 30/70, 0.22/0.51 g/l in the normal serum pool (500 blood donors). In this study the G1m(a)/G1m(f) was adjusted to 33/gG according to the IgG1 levels found in homozygous genotypes, respectively, referring to a content in the normal serum pool of 1.73 g/l for G1m(a) and 3.34 g/l for G1m(f). Mean +/−SD of G1m(f), G1m), G2m(n) and G3m(b) are given in per cent of a normal serum pool =100% and in g/l. In serum samples, the sensitivity of the ELISA assay was for G1m(a) 0.0008 g/l, for G1m(f) 0.0003 g/l, for G2m(n) 0.0006 and for G3m(b) 0.0007 g/l.

For homozygous G2m(-n-n) and G3m(gg), the levels are equal to the IgG2 and IgG3 levels, respectively The heterozygous G2m(n-n) individuals were determined with murine monoclonals anti-G2m(n) 6016-10 clone SH-21 and anti-IgG2 HP 6014 (Sigma) in a double immunodiffusion assay. For heterozygous 2m(n-n), the G2m(-n) level was calculated by subtraction of the G2m(n) amount from the IgG2 amount and for heterozygous G3m(gb) the G3m(g) level was calculated by subtraction of G3m(b) from IgG3. G2m(-n) and G3m(g) are only given as g/l.

The serum concentrations of IgG1, IgG2, IgG3 and IgG4 were determined with the radial immunodiffusion technique and specific polyclonal rabbit antisera. Mean and SD have been given for the different age groups of the six most common Gm genotypes, respectively. IgG subclass levels were also given for all children of the age groups as mean and SD. A normal serum pool of 500 blood donors was used as reference serum containing: IgG1, 6.48 g/l; IgG2, 3.41 g/l; IgG3, 0.67 g/l and IgG4 0.46 g/l according also to the WHO reference serum 67/97.

A summary of an example of a competitive ELISA method for qualitative investigation of the Gm haplotypes and genetic B cells and for quantitative analyses of different Gm allotypes is as follows:

1) Coating with purified myeloma proteins of type

G1m(f)

G1m(a)

G2m(Cn)

G3m(b), respectively

2) Adding monoclonal antibodies of type

Anti-G1m(f)

Anti-G1m (a)

Anti-G2m (n)

Anti-G3mn(b) r respectively

3) Adding patient's serum

4) Adding dilutions of a normal serum pool.

### Example 2

### Application In Clinical Practice

1) In Bronchial Asthma

Impaired lung function was detected in every third IgA deficient patient with recurrent infections occuring predominantly in association with patients with additional low levels of IgG2 or IgG3. Prophylaxis with a immunoglobulin preparation low in IgA was suggested to prevent lung damage in these patients.

An investigation of 6 580 patients (4716 children) with recurrent chronic or severe infections exposed 186 with IgG3 deficiency. The most common diagnosis was infectious prone bronchial asthma together with recurrent sinuitis and otitis.

The increased frequency of G2m(n) allotype in atopic patients and the preponderance of the Gm haplotype Gm(bfn) established that a genetic IgG constitution is related to atopy. The G2m(n) allotype was found in 76% of 50 patients with IgE>600kU/l, in 88% of 25 patients with IgE>1000kU/l and in 94% of 16 patients with IgG4>1g/1.

Patients with increased IgG4 together with increased IgE are known to have more severe manifestations of their atopic disease. The imbalanced IgG subclass levels of 50 atopic patients with IgE>600 kU/l reflected the Gm expression of the patients. The influence of Gm allotype gene dosage on IgG subclass levels was also clear.

When comparing two groups of children with bronchial asthma, one group with increased IgE and specific IgE antibodies to different allergens and the other with low IgE and abscence of specific IgE antibodies, it was found that the former group had the Gm(bfn/bfn) genotype as expected while the other had the Gm(ga-n/ga-n) genotype with the alternative Gm allotypes. The experience was that patients with the Gm(ga-n/ga-n) genotype never developed specific IgE antibodies against allergens. The investigation of G2m(-n) could be of diagnostic help already early in wheezing disease.

A group of people who on exposure had developed laboratory animal allergy was examined. This group had been thoroughly examined by others. Those who developed allergy, shown by symptoms and specific IgE antibodies, was mainly of the Gm(bfn/bfn) genotype, once again showing the connection of increased IgE, specific IgE and particular Gm genes.

When children with bronchial asthma selected in two groups with the alternative Gm allotypes of IGHG genes were investigated, it was found that the Gm(bfn/bfn) genotype expressing IgG1 molecules with the characteristics of G1m (f), IgG2 as G2m(n), IgG3 as G3m(b) also showed the phenotype with increased IgE and specific IgE, increased number of peripheral eosinophils, low number of CD 8 lymphocytes, the atopic phenotype. The other asthma group with the Gm(ga-n/ga-n) genotype expressing IgG1 molecules as G1m(a), IgG2 as G2m(-n) and IgG3 as G3m(g) showed the characteristics of the non atopic form of bronchial asthma with low IgG3(G3m(g) levels.

The alternative G1m, G2m and G3m allotypes of IGHG genes correlate with the atopic and the non atopic pathways of immune regulation in children with bronchial asthma. While the G2m(n) allotype can be used to further confirm atopy in children, the G2m(-n) allotype can be used to put a diagnosis on non atopy, e.g. small children with asthma or recurrent wheezing. This is ready to be used in the clinic.

The four human Gm haplotypes reflect different characteristics of B cells among patients with bronchial asthma. The IgG expression on gene level has not been investigated before among children with bronchial asthma. The finding of particular Gm allotypes in atopy and the alternative Gm allotypes in infectious bronchial asthma is extremely important and will make it possible to understand these conditions better. This study will be the basis for investigating patients with beside bronchial asthma also other forms of allergy and hypersensitivity as eczema, hay fever, food allergy, urticaria and anaphylaxis. Gm allotypes will be outstanding markers for different form of allergy.

Quantitation of different Gm allotypes in bronchial asthma and development of a simple method for screening non-atopic bronchial asthma is in progress. The association of particular Gm allotypes with different severe forms of bronchial asthma will be of help in making prognosis and finding risk patients.

The invention may be used to further investigate patients with bronchial asthma and patients with immunodeficiencies suffering from obstructive lung disease. The non atopic form has similarities with the immunodeficiencies, unfortunately not often diagnosed as such. Screening for G2m(-n) is of help when evaluating patients with low IgE and no specific IgE.

2) In Immunodeficiencess and Autoimmune Diseases

It has now been shown that IgG3 deficiency is associated with the G3m(g) allotype. The relevance of G2m(n) allotype for the response to bacterial polysaccharide antigens and for the level of specific polysaccharide IgG2 antibodies has been documented. A complete Gm allotyping of both IgG2 and IgG3 deficient patients revealed lack of G2m(n). The IgG2 deficient patients were homozygous Gm(bf-n/bf-n) and the IgG3 deficient patients were homozygous Gm(ga-n/ga-n). The IgG2 levels differed if the G2m(-n,-n) was associated with G1m(f,f) or G1m(a,a) on IgG1 locus, and G3m(b,b) or G3m(g,g) on IgG3 locus. The G2m(-n,-n) could be designated a low antibody response gene. There was evidence that the level of one IgG subclass is decided not by one Gm allotype alone, but by the whole Gm haplotype, probably influenced by a regulator gene.

The results also indicate that IgG2 deficiency and IgG3 deficiency are genetically heterogenous groups. The homozygous Gm gene expression in IgG2 and IgG3 deficient patients confirm the more restricted IgG molecules earlier seen in serum electrophoresis in IgG2 deficiency as short anodal distribution and in IgG3 deficiency as short cathodal distribution.

Gm allotyping should be used in IgG subclass deficient patients for further evaluation of their immune capacity. The deviating electrophoretic distribution of alternative Gm allotypes within the same IgG subclass has been shown.

There was a linkage of Gm allotypes to IgA deficiency, especially homozygous G2m(-n, -n) on IgG2 locus. This favours the combination of IgA deficiency and IgG subclass deficiency. The quality of IgG molecules is restricted with G1m(a) as IgG1 molecules, G2m (-n) as IgG2 molecules and G3m(g) as IgG3 molecules, in spite of the ‘compensatory increased’ IgG1 and IgG3.

The gene locus for IgG3 and IgG1 is situated upstream and the gene locus for IgG2 and IgG4 downstream of IgA1 on the q32 band of chromosome 14. The activities from the IgG3 and IgG1 loci were more pronounced than from the downstream located IgG2 and IgG4. This influences the quality and quantity of specific IgG antibodies in IgA deficient individuals. It speaks for a down regulated part of the IGHG gene.

When quantitating Gm allotypes in IgA deficient individuals the very rare G2m(n) allotype on IgG2 locus was expressed only to about 50%. The G2m(-n) also from the same IgG2 locus was expressed with normal amounts of IgG2 antibodies. This could be the result of allotype suppression earlier described in mice but not yet in man and no anti-Gm antibodies were found. The IgA1 gene might have a suppressive influence on the G2m(n) allotype but not on the G2m(-n) allotype on chromosome 14, exposed to the same regulating mechanism on the gene. A defect switching in some cells or retardation from IgA1 locus and downstream of the chromosome 14 g32 when the IgG2 locus is expressed with an G2m(n) allele can be suggested as the mechanisms involved

Common variable immunodeficiency is another frequent primary immunodeficiency with development of chronic obstructive lung disease. 25 of 33 expressed homozygous G2m(-n) (p<0.001) on IGHG2 locus with a progressive impediment production of the Gm allotype molecules G1m(a) more than G1m(f) downstream the chromosome 14q32.

IGHG genes have an influence on different forms of disease as in Juvenile Chronic Arthritis (JCA). In the most severe form, the systemic JCA, the Gm(ga-n/ga-n) genotype was common. Instead, patients with the Gm(bfn/bfn) genotype had the best prognosis and characteristically increased IgG3 and IgG4, but this genotype was significantly rare among JCA patients. Patients with JCA, especially the polyarticular subsets, most often expressed their IgG1 as G1m(a) molecules, their IgG2 as G2m(-n) molecules and their IgG3 as G3m(g)molecules, which must be considered in the pathogenesis of JCA. Determination of the Gm genotypes could be helpful indicators in judging prognosis and risk in JCA.

1. A method for in vitro determination of allelic alternative genetic markers for the IgG subclasses IgG1, IgG2 and IgG3 from chromosome 14q32 in serum of a patient, wherein the allelic expression of the activated IGHG1, IGH2 and IGHG3 genes is determined.

2. A method according to claim 1, wherein the Gm allotypes G1m(f), G1m(a), G2m(n), Gm2(-n), G3m(b) and Gm3 (g) are determined resulting in the classification of four different Gm haplotypes, i.e. Gm(bfn), Gm(bf-n), Gm(gan) and Gm(ga-n), and four types of genetically different B cells, i.e. B1, B2, B2 and B4, respectively.

3. A method according to claim 1, wherein B1 cells with the genetic code Gm(bfn) and with G2m(n) on IgG2 locus in linkage disequilibrium with a high producing IgE gene are determined.

4. A method according to claim 1, wherein B2 and B4 cells with the genetic code Gm(bf-n) and Gm(ga-n), respectively, both with G2m(-n) on IgG2 locus in linkage disequilibrium with a low producing IgE gene are determined.

5. A method according to claim 1, wherein the determination of the allelic alternative genetic markers G2m(-n) and G3m(g) is combined with a determination of IgG subclasses IgG2 and IgG3, respectively.

6. A method according to any one of claims 1-5, wherein the determination is quantitative.

7. A method according to claim 1, wherein the patient is human.

8. A method according to claim 1, which method is a method for diagnosis or research of allergic disease.

9. A method according to claim 8, wherein the allergic disease is bronchial asthma.

10. A method according to claim 8, which is a method of differentiating between atopic and non atopic allergic disease.

11. A method according to claim 8, wherein the prognosis of allergic disease is determined by determination of homozygous variants of B cells.

12. A method according to claim 1, which is a method for diagnosis and/or research of immunodeficiencies.

13. A method according to claim 1, which is a method of diagnosis and/or research of autoimmune diseases.

14. A method according to claim 1, which is an enzyme linked immunosorbent assay (ELISA-method), preferably of competitive type.

15. A kit for in vitro determination of genetic markers for the IgG subclasses IgG1, IgG2 and IgG3 from chromosome 14q32 in serum of a patient, comprising
i) a set of purified myeloma proteins of the Gm allotypes G1m(f), G1m(a), G2m(n) and G3m(b),
ii) a set of monoclonal anti-G1m(f)-, anti-G1m(a)-, anti-G2m(n)- and G3m(b)-antibodies, respectively,
iii) a control panel of purified myeloma proteins of the Gm allotypes G1m(f), G1m(a), G2m(n) and G3m(b).
